(19) United States
(12) Patent Application Publication
(10) Pub. No.: US 2022/0413136 A1
(43) Pub. Date: Dec. 29, 2022

(54) ULTRASOUND IMAGING DEVICE AND METHOD OF GENERATING COLOR DOPPLER IMAGE

(71) Applicant: FUJIFILM Healthcare Corporation, Kashiwa-shi (JP)

(72) Inventors: Tomohiko Tanaka, Chiba (JP); Tetsuya Yamada, Chiba (JP); Hideki Yoshikawa, Chiba (JP)

(21) Appl. No.: 17/832,915

(22) Filed: Jun. 6, 2022

(30) Foreign Application Priority Data
Jun. 24, 2021 (JP) 2021-105174

(51) Int. Cl. G01S 15/89; A61B 8/06; A61B 8/08

(52) U.S. Cl. CPC G01S 15/8979; A61B 8/06; A61B 8/488; A61B 8/5207; A61B 8/13

(57) ABSTRACT

A clutter signal mixed in a blood flow signal is reduced in a color Doppler, and blood flow visibility is improved. A combination of parameters that maximize a difference between a blood flow and a clutter (a signal other than the blood flow) is determined by analyzing a reception signal, a clutter estimated value (a value indicating a degree of being estimated as a clutter) is set based on the combination, and a reduction coefficient map (hereinafter, simply referred to as a reduction map) that reduces a clutter signal is generated based on the estimated value. The clutter signal is reduced by multiplying the reception signal (an IQ signal after quadrature detection) by the reduction map.

### BACKGROUND OF THE INVENTION

### 1. Field of the Invention

The present invention relates to an ultrasound imaging device, and more particularly to a technique for removing a clutter component caused by a body motion in a color Doppler image.

### 2. Description of the Related Art

Color Doppler imaging is an imaging method of displaying a two-dimensional distribution of a blood flow velocity in a heart or a blood vessel in color superimposed on a tissue tomogram by using a Doppler effect of an ultrasonic wave reflected from a moving object (mainly blood) included in an inspection target, and is widely applied as blood flow display of an organ in ultrasonography.

An ultrasonic signal reflected from the inspection target includes not only a signal from a blood flow but also an unnecessary reflection signal from a tissue that moves at a speed slower than that of the blood flow, such as a movement of a cardiac wall. Such an unnecessary reflection signal is called a clutter. Since mixing of a clutter component inhibits blood flow visibility, in the color Doppler imaging, in order to remove the clutter component from a received ultrasonic signal, a low-pass filter called a wall filter, a moving target indicator (MIT), a clutter removing filter, or the like is generally used.

A low-pass filter in the related art is designed based on Doppler shift by frequency analysis on the basis of a fact that the clutter component is a reflection signal from a tissue having a low speed.

In application of the filter, it is necessary to effectively remove the clutter component without reducing the reflection signal from the blood flow, and various proposals are made for the low-pass filter. For example, JP-A-2014-8076 discloses a technique of adjusting a cutoff characteristic of a filter to match a clutter component by dynamically shifting a frequency axis in filter processing based on a variance value of a signal, and discloses obtaining power based on a signal before the filter processing and changing an index for determining a coefficient of the filter in consideration of the power.

On the other hand, since the received ultrasonic signal includes noise (electric noise or the like) other than the clutter component, a technique of executing processing based on characteristics of the noise or the like on a color Doppler image generated after the processing executed by the clutter removing filter (JP-A-2014-8076 described above, JP-A-2012-110706, or the like) is also proposed.

### SUMMARY OF THE INVENTION

Clutter removal by a low-pass filter in the related art can effectively remove tissue clutter that moves at a speed slower than the blood flow velocity. However, a characteristic of a clutter removing filter is that a component having a frequency equal to or higher than a certain frequency passes through the clutter removing filter, and cannot remove, for example, a component caused by a body motion of a subject under imaging. When such a body motion component is included as a residual clutter, it is difficult to distinguish the residual clutter from a blood flow signal, and in a color Doppler image generated based on a signal after the filter processing, the residual clutter is obvious, and the blood vessel visibility is inhibited.

An object of the invention is to provide means for effectively removing a signal other than the blood flow signal, such as a body motion, which cannot be removed by a clutter filter in the related art, and acquiring an image having good blood vessel visibility.

According to the invention, a feature that maximizes a difference between a blood flow and a clutter (a signal other than the blood flow) is determined by analyzing a reception signal, a clutter estimated value (a value indicating a degree of being estimated as a clutter) is set based on the feature, and a reduction coefficient map (hereinafter, simply referred to as a reduction map) that reduces a clutter signal is generated based on the estimated value. The clutter signal is reduced by multiplying the reception signal (an IQ signal after quadrature detection) by the reduction map.

That is, an ultrasound imaging device according to the invention includes: a transmission and reception unit configured to transmit and receive an ultrasonic wave; a clutter processing unit configured to remove a clutter signal from a reception signal of the ultrasonic wave received by the transmission and reception unit; and a color Doppler calculating unit configured to generate a color Doppler image using the reception signal from which the clutter signal is removed. The clutter processing unit includes a feature detecting unit configured to detect a feature of the reception signal, a determining unit configured to determine, based on the feature, whether the reception signal is a blood flow signal or a clutter signal, a reduction map generating unit configured to generate, based on a determination result, a reduction map for reducing the clutter signal, and a clutter reducing unit configured to apply the reduction map to the reception signal. The clutter processing unit may further include a clutter filter.

In addition, a method of generating a color Doppler image according to the invention includes: a clutter processing step of removing a clutter signal from a reception signal; and a color Doppler calculating step of generating a color Doppler image using the reception signal from which the clutter signal is removed. The clutter processing step includes a feature detecting step of detecting a feature of the reception signal, a determination step of determining, based on the feature, whether the reception signal is a blood flow signal or a clutter signal, a reduction map generating step of generating, based on a determination result, a reduction map for reducing the clutter signal, and a clutter reducing step of applying the reduction map to the reception signal.

According to the invention, before or after the clutter filter is applied, the reception signal is multiplied by the reduction map generated using an index (a feature) that maximizes the difference between the blood flow signal and the clutter signal, whereby a filter function based on frequency analysis is compensated, the clutter signal including a body motion other than the tissue clutter can be effectively reduced, and a color Doppler image having good blood vessel visibility can be provided.

### BRIEF DESCRIPTION OF THE DRAWINGS

FIG. 1 is a block diagram illustrating an ultrasound imaging device according to an embodiment of the invention.

FIG. 2 is a block diagram illustrating a configuration of a clutter processing unit in FIG. 1.

FIG. 3 is a diagram illustrating a flow of color Doppler image generation executed by the ultrasound imaging device according to the embodiment.

FIG. 4 is a diagram illustrating a flow of processing of a clutter reducing unit according to a first embodiment.

FIGS. 5A and 5B are diagrams illustrating cutoff setting by histogram analysis as examples of parameters.

FIG. 6 is a diagram illustrating an example of a screen for user setting.

FIG. 7 is a diagram illustrating an example of a clutter reduction map.

FIG. 8 is a diagram illustrating an effect according to the embodiment.

FIG. 9 is a diagram illustrating a flow of processing according to a modification of the first embodiment.

### DESCRIPTION OF EMBODIMENTS

Hereinafter, an ultrasound imaging device according to an embodiment of the invention will be described with reference to the drawings.

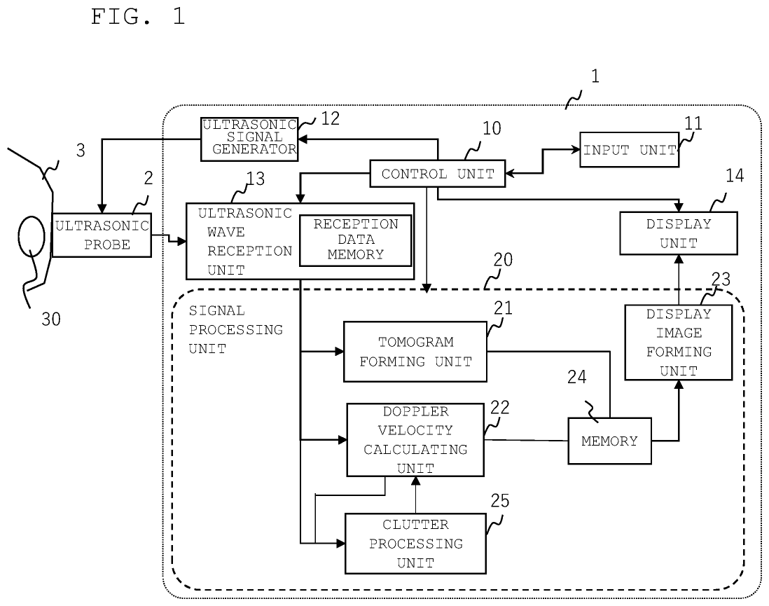

As illustrated in FIG. 1, an ultrasound imaging device 1 according to the present embodiment includes: an ultrasonic signal generator 12 and an ultrasonic wave reception unit 13, as an ultrasonic wave transmission and reception unit, to which an ultrasonic probe 2 is connected; a signal processing unit 20 that executes various types of signal processing and calculation on an ultrasonic signal (a reception signal) received by the ultrasonic wave reception unit 13; and a control unit 10 that controls operations of the ultrasonic wave transmission and reception unit and the signal processing unit 20. The ultrasound imaging device 1 may further includes an input unit 11 for inputting information, a condition, a command, and the like, which are necessary for the signal processing and control, to the control unit 10 and a display unit 14 that displays an ultrasonic image or the like being a processing result of the signal processing unit 20.

The ultrasonic probe 2 is a device that transmits and receives an ultrasonic wave by being pressed against a subject 3, and examples thereof include various types of ultrasonic probes that differ depending on an imaging method and an imaging target. Although the ultrasonic probe 2 is not particularly limited, a general array probe in which a large number of piezoelectric elements are arranged in a one-dimensional direction or a two-dimensional direction can be used.

A function of the ultrasonic wave transmission and reception unit is the same as that of a general ultrasound imaging device. The ultrasonic signal generator 12 generates an ultrasonic pulse having a predetermined frequency (a transmission frequency) and transmits the ultrasonic pulse to each element of the ultrasonic probe 2 at a predetermined timing. The ultrasonic wave reception unit 13 includes a phasing unit and an A/D converting circuit (which are not illustrated) and a reception data memory, and executes phasing for each frame, stores a reception signal after A/D conversion in the reception data memory, and transmits the reception signal to the signal processing unit 20.

The signal processing unit 20 includes: a tomogram forming unit 21; a Doppler velocity calculating unit 22; a display image forming unit 23; a memory 24; and a clutter processing unit 25 that executes processing for clutter reduction on the reception signal. Functions of the tomogram forming unit 21, the Doppler velocity calculating unit (a color Doppler calculating unit) 22, and the display image forming unit 23 are the same as those of the general ultrasound imaging device. Briefly, the tomogram forming unit 21 receives a reception signal for each frame from the reception data memory of the ultrasonic wave reception unit 13, and sends the reception signal as a packet signal to the display image forming unit 23. A packet is a data string of reflected encoder signals from the same location (the same depth) of data irradiated a plurality of times in the same direction. The display image forming unit 23 includes a digital scan converter (DSC), and generates, by using a digital signal from the tomogram forming unit 21, a tomogram (a B-mode image) to be displayed on the display unit 14.

The Doppler velocity calculating unit 22 includes a quadrature detector, an autocorrelator, and the like (which are not illustrated), calculates a Doppler shift amount, a blood flow velocity and a variance, a blood flow amplitude intensity, and the like using an IQ signal after quadrature detection (an in-phase signal/quadrature phase signal), determines, based on the Doppler shift amount, a blood flow velocity and direction (an approaching direction or a moving away direction) with respect to a probe, and sends results thereof to the display image forming unit 23. The display image forming unit 23 generates a color Doppler image by superimposing information relating to the blood flow velocity calculated by the Doppler velocity calculating unit on the B-mode tomogram. The memory 24 is used to temporarily store a signal from the tomogram forming unit 21 and a tomogram for each frame generated based on the signal.

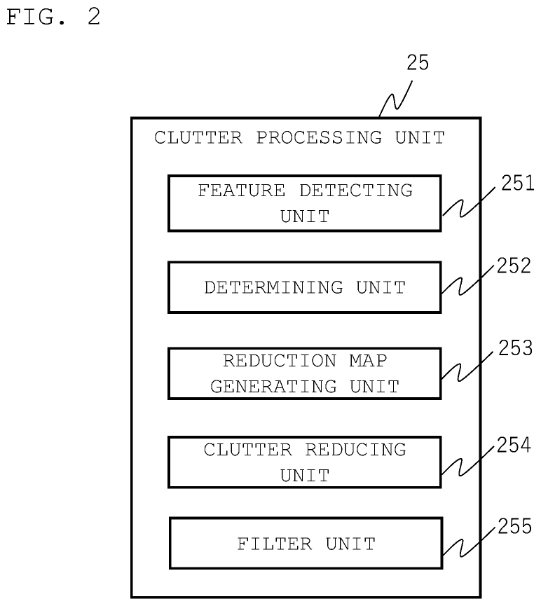

The clutter processing unit 25 executes processing necessary for removing a clutter component by using the IQ signal after the quadrature detection input to the signal processing unit 15 or intermediate data of the Doppler velocity calculating unit 22. Therefore, as illustrated in FIG. 2, the clutter processing unit 25 includes a feature detecting unit 251 configured to detect a feature of the reception signal, a determining unit 252 configured to determine, based on the feature, whether the reception signal is a blood flow signal or a clutter signal, a reduction map generating unit 253 configured to generate, based on a determination result, a reduction map for reducing the clutter signal, a clutter reducing unit 254 configured to apply the reduction map to the reception signal, and a filter unit 255.

A feature to be detected by the feature detecting unit 251 is a parameter representing a physical difference between the clutter and the blood flow, and is, for example, a feature directly calculated based on the packet signal such as an amplitude or a standard deviation (a standard deviation of a certain depth in a time direction) of the IQ signal in a packet direction, or an average velocity or a velocity variance of the blood flow velocity calculated by the Doppler velocity calculating unit 22. The feature detecting unit 251 analyzes a signal value of the packet signal or intermediate data such as a Doppler velocity calculated by the Doppler velocity calculating unit 22, and calculates a feature for identifying the clutter and the blood flow.

The determining unit 252 determines, regarding features calculated by the feature detecting unit 251, a threshold value of a feature that maximizes a difference between the blood flow and the clutter (a signal other than the blood flow) in a reception signal to be processed.

The reduction map generating unit 253 calculates a clutter estimated value as a ratio of estimating a signal as a clutter signal using the threshold value of the feature determined by the determining unit 252, and determines a reduction map having the clutter estimated value as a coefficient. The clutter estimated value may be determined using only one feature, and is preferably calculated by combining a plurality of features. Accordingly, as compared with the case of using one feature, an accuracy of the reduction map is improved, that is, an accuracy of identifying the clutter and the blood flow is improved.

The reduction map to be generated is determined as a map of a space corresponding to an image space of the B-mode image. The clutter reducing unit 254 multiplies the packet signal by the reduction map, and reduces a clutter component in the packet signal.

The filter unit 255 is not essential when an accuracy of clutter reduction processing executed by other units of the clutter processing unit 25 is high, but is applied to an output of the clutter reducing unit 254. The filter unit 255 includes a low-pass filter such as a known wall filter or MIT filter, and removes a clutter in the data processed by the clutter reducing unit 254. The processing by the filter unit 255 may be executed at a stage before the feature detecting unit 251.

A part of or all functions of the above-described signal processing unit 20 can be implemented by a computer equipped with a memory and a CPU or a GPU, and is executed by uploading to the computer a program prepared in advance to implement the function of each unit. A part of the functions of the signal processing unit 20 may be implemented by hardware such as an application specific integrated circuit (ASIC) or a field programmable gate array (FPGA). The clutter processing unit 25 may include a learning model such as a learned convolutional neural network (CNN).

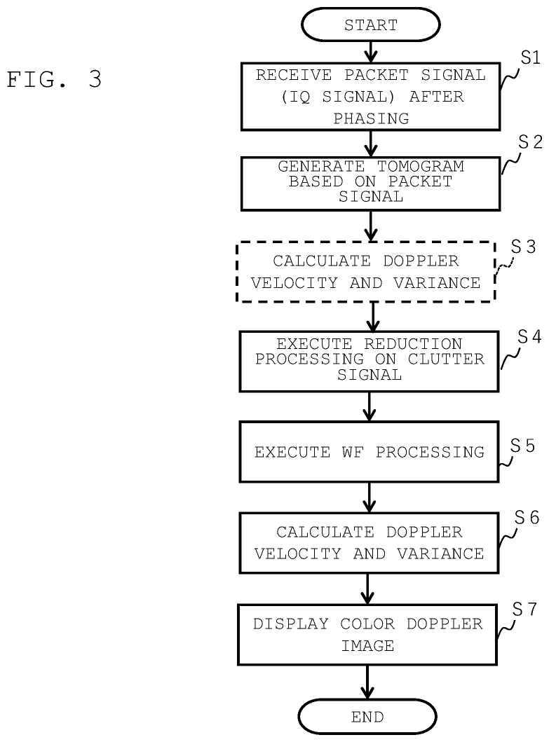

An outline of operations during color Doppler imaging executed by the ultrasound imaging device according to the present embodiment is illustrated in FIG. 3.

First, the ultrasonic probe 2 is brought into contact with a body surface of the subject 3, and packet transmission and reception are repeated while a tissue or an organ, which is an inspection target 30, is scanned with the ultrasonic probe 2 at a predetermined angle. A reflected wave from the inspection target 30 is detected by the ultrasonic probe 2, is subjected to phasing by the ultrasonic wave reception unit 13, is then stored in the reception data memory for each frame, and is then sent to the tomogram forming unit 21, the Doppler velocity calculating unit 22, and the clutter processing unit 25 (S1).

The tomogram forming unit 21 forms a tomogram (a B-mode image) using the data for each frame, stores the tomogram in the memory 24, and sends the tomogram to the display image forming unit 23 via the memory 24 (S2). The display image forming unit 23 converts the tomogram received from the memory 24 into an image to be displayed on the display unit 14. Accordingly, the image of the inspection target 30 is displayed on the display unit 14 at a predetermined time resolution.

On the other hand, the Doppler velocity calculating unit 22 executes quadrature detection on the packet signal to convert the packet signal into an IQ signal, then executes frequency analysis for each position based on a correlation between packet signals, and calculates a velocity (S3). Since the velocity calculation is executed on a signal that has not been subjected to filter processing, velocity information includes not only the blood flow velocity but also a blood vessel wall velocity, a velocity based on the body motion, and the like. The velocity information calculated by the Doppler velocity calculating unit 22 before the filter processing is referred to as the intermediate data. However, in a case where the velocity information is not used for calculation of a feature to be described later, the processing S3 is omitted.

The clutter processing unit 25 receives the IQ signal after the quadrature detection and the above-described intermediate data from the Doppler velocity calculating unit 22, and first executes the clutter reduction processing (S4).

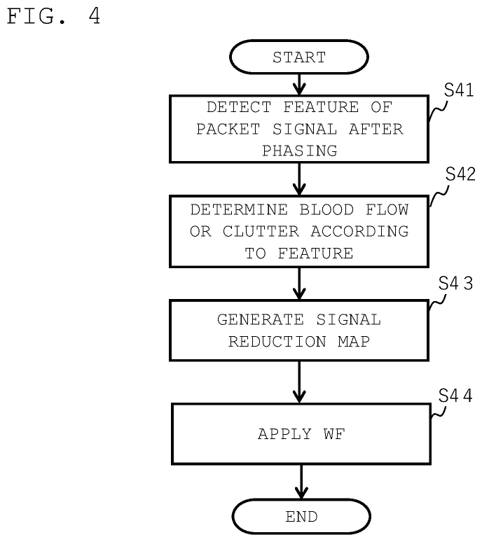

In the clutter reduction processing, as illustrated in FIG. 4, first, the feature detecting unit 251 detects, based on the signals or the data, a feature for determining a blood flow and a clutter (S41). The feature may be a single feature, or may be detected for each of a plurality of items (for example, an amplitude, a velocity, a variance, Or a standard deviation). The determining unit 252 determines whether one or each of a plurality of features is a clutter (S42), and the reduction map generating unit 253 combines the features to generate a map of clutter estimated values based on a clutter determination result (S43). The map is a map representing the clutter estimated values at respective positions where the packet signal is obtained, and may take a binary value of 1 or 0 regarding whether the packet signal is a clutter, or may take an intermediate value between 0 and 1 when the determination includes a gray zone.

The clutter reducing unit 254 applies the map of the clutter estimated values to data (an IQ signal of the packet) received from the reception data memory, and obtains data of which the clutter is reduced (S44). Thereafter, the clutter is removed by the filter unit 255 (S5).

The data processed by the above-described clutter processing unit 25 is passed to the Doppler velocity calculating unit 22 where calculation is executed using phase information of the IQ signal after the clutter signal is reduced, and the Doppler velocity, the variance thereof, and the like are calculated (S6). The calculated blood flow velocity information is input to the display image forming unit 23 as color Doppler information to which different colors are added depending on an angle with respect to a direction of a transmitted ultrasonic signal, is converted into a color Doppler image superimposed on the tomogram in the display image forming unit 23, and is displayed on the display unit 14 (S7).

In the ultrasound imaging device according to the present embodiment, the clutter and the blood flow are identified from each other based on the IQ signal and data (the velocity information) during processing in the Doppler velocity calculating unit 22, a clutter reduction map for reducing only the clutter is generated, and the clutter reduction map is applied to the original signal. Whereby, an accuracy of the clutter removal can be increased, a clutter component that is likely to be mixed in the body motion or the blood flow can be reduced, and the blood flow visibility can be greatly improved, as compared with the processing using only the low-pass filter based on a Doppler frequency.

Next, the processing of the clutter processing unit will be described using a specific example of the feature.

### First Embodiment

In the present embodiment, a case will be described in which a variance value of the blood flow velocity and an IQ standard deviation are used as features for identifying the clutter and the blood flow.

First, the feature detecting unit 251 calculates, as features, a standard deviation of signal values (amplitudes) of the IQ signal and the variance value of the blood flow velocity. An IQ standard deviation value σ is calculated based on the IQ signal received by the clutter processing unit 25. A velocity variance value is the variance value of the blood flow velocity (a velocity at each position) calculated based on the IQ signal, and the Doppler velocity calculating unit 22 calculates the variance value in accordance with calculation of the blood flow velocity based on the IQ signal after the quadrature detection. The feature detecting unit 251 receives, as the intermediate data, the variance value of the blood flow velocity calculated by the Doppler velocity calculating unit 22 before clutter processing, and uses the variance value as a feature.

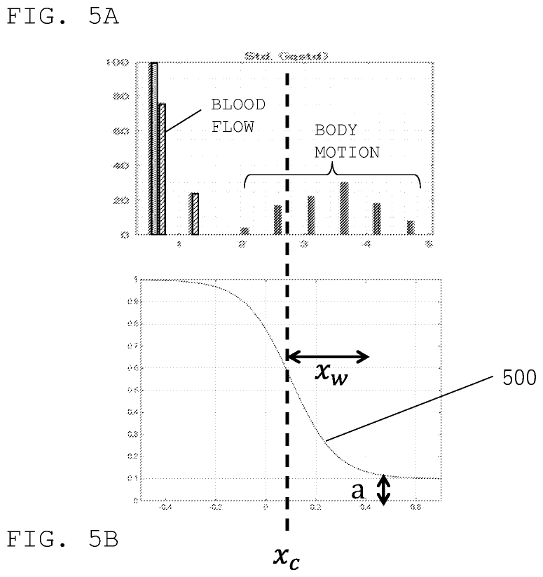

Next, the determining unit 252 executes histogram analysis on the IQ standard deviation value σ and the velocity variance value, and sets a threshold value for distinguishing organ parenchyma (tissue) as an inspection target and a clutter caused by a body motion by using a method such as a determination analysis method. FIG. 5A illustrates an example of a histogram. Here, FIG. 5A illustrates, when the IQ standard deviation value σ and the velocity variance value are generalized and set as measured values x, a histogram of the measured values x. As illustrated in FIG. 5A, the signal values of the organ parenchyma (tissue) as an inspection target and the blood flow appear with a high frequency, but a clutter signal appears in a dispersed manner in a low frequency region. In order to reduce a body motion signal, the determining unit 252 generates a reduction filter 500 in which a value for distinguishing the organ parenchyma and the clutter is set as a cutoff value. As the reduction filter, a function that monotonically decreases and monotonically increases with a threshold value as a boundary can be adopted, and here, the reduction filter is modeled by a sigmoid function represented by the following equation (1). FIG. 5B illustrates an example of the reduction filter 500 using the sigmoid function.

$$y = 1 - \frac{( 1 - a )}{1 + exp [ - \frac{2.2}{{x}_{w}} ( x - {x}_{c} ) ]}$$

In the equation, x represents a measured value, x_{c} represents the cutoff value, x_{w} represents a width (a width allowing overlap between the body motion and other portions), and “a” represents an offset (an offset for preventing tissue and blood flow signals in a low frequency from being removed).

As the cutoff value, a threshold value determined based on the histogram can be used. The width x_{w} and the offset “a” can be set to predetermined values in advance based on an experience value, a simulation using a phantom, or the like. However, since characteristics of the reduction filter 500 differ depending on a setting method thereof, the width x_{w} and the offset “a” may be set according to characteristics desired by a user. For example, when the width x_{w} or the offset “a” is decreased, a degree of reduction increases, but a possibility of reducing a signal that should not be reduced is also increased. On the other hand, when the width or the offset is increased, there is a possibility that the body motion is not sufficiently reduced. The characteristics may be represented as, for example, “Low”, “Mid”, or “High” according to values of the width x_{w} and the offset “a”, and the user may select a desired characteristic via the display unit 14.

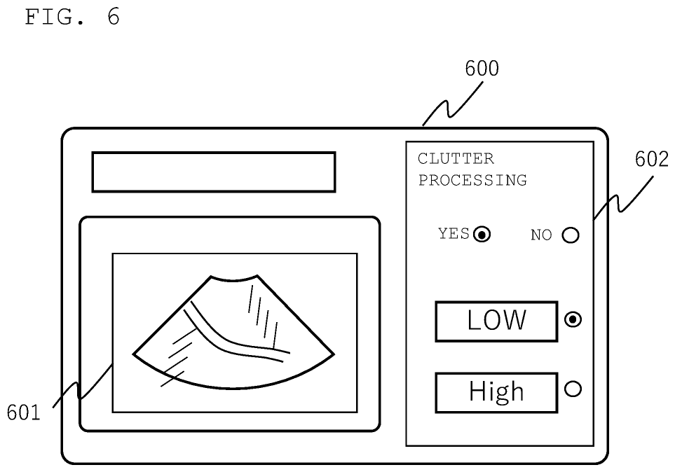

FIG. 6 illustrates an example of a display screen. In this example, for example, a color Doppler image 601 after the clutter removal processing executed by the filter unit 255 is displayed, and a block 602 indicating a degree of processing (“High” or “LOW”) executed by the clutter processing unit 25 is displayed. The user observes the color Doppler image 601 to confirm a state in which the blood flow visibility is inhibited by the body motion, and selects a degree of reducing the body motion. FIG. 6 is an example in which the color Doppler image after the processing executed by the filter unit 255 is displayed. Alternatively, as a display image, the color Doppler image, the B-mode image, and the like before the processing executed by the filter unit 255 may be displayed, or only a display of the block 602 may be displayed.

An identification model adopted by the determining unit 252 is not limited to the sigmoid function shown by the equation (1), and any function that monotonically decreases and monotonically increases with a threshold value as a boundary may be adopted. For example, a Step function, an Error function, or the like can be adopted.

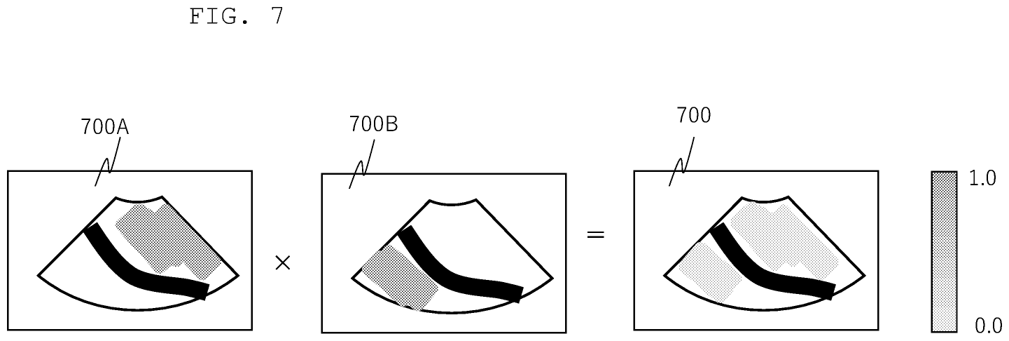

Next, the reduction map generating unit 253 generates a final clutter reduction map using reduction filters generated for the plurality of features, here, the IQ standard deviation and the velocity variance value. A final clutter reduction map 700 has, for example, coefficients from 0 to 1 at the respective positions, and may be obtained by simply multiplying a plurality of reduction filters 700A and 700B as illustrated in FIG. 7, or may be obtained by weighting.

The clutter reducing unit 254 applies the clutter reduction map 700 to the IQ signal received by the clutter processing unit 25, and obtains data after clutter reduction. When a known wall filter is applied as the filter unit 255 for the data, the processing executed by the clutter processing unit 25 is completed.

### Effects of First Embodiment

An effect when the clutter reduction processing according to the first embodiment is assumed to be applied to liver imaging will be described. Parameters (parameters for determining the sigmoid function) of the reduction filter of each feature generated by the determining unit 252, that is, the cutoff value and the width of the standard deviation of the IQ signal can be set to values corresponding to a sensitivity level of the IQ signal. The offset value is a value of 0 to 1. Here, reduction filters of “Low” and “High” are generated with different parameter values.

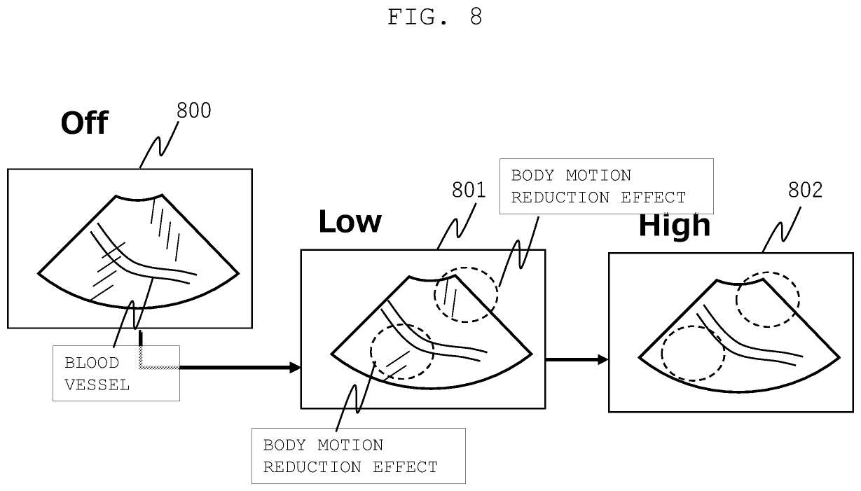

Images obtained by the imaging are schematically illustrated in FIG. 8. The left image in FIG. 8 illustrates a case where only normal WF processing is executed without executing the clutter reduction (800). The middle and right images in FIG. 8 illustrate cases where the clutter reduction processing is executed according to the processing of the present embodiment before the WF processing. The middle image illustrates a reduction filter of “Low” (801), and the right image illustrates a reduction filter of “High” (802).

From the images illustrated in FIG. 8, it can be confirmed that, by executing the reduction processing according to the present embodiment by confirming an improvement of the blood vessel visibility realized by a clutter reduction effect, the body motion signals, which are insufficiently reduced by a method in the related art, are reduced without reducing the blood flow signal.

As is clear from the above description, according to the present embodiment, characteristics of the IQ signal itself to be used for Doppler velocity calculation are analyzed, and a clutter component included in the IQ signal is reduced using the feature for identifying the blood flow signal and the body motion clutter, whereby a clutter component that cannot be removed by the filter based on the Doppler frequency can be effectively reduced. In addition, by combining the clutter reduction processing and a known clutter filter, a color Doppler image having excellent blood flow visibility can be displayed.

In addition, according to the present embodiment, by using the reduction map generated by combining a plurality of features, it is possible to execute reduction processing corresponding to various body motions having different intensities and frequencies. Further, according to the present embodiment, by displaying a selection screen of the degree of reduction, the user can determine and select the degree of reduction by viewing the image, and therefore highly effective reduction processing can be executed even for images of different inspection targets and imaging conditions.

### Modification of First Embodiment

In the first embodiment, in order to generate the reduction map, two features including the standard deviation of the IQ signal and the velocity variance are used in combination. Alternatively, only one of the standard deviation of the IQ signal and the velocity variance may be used. When one of the features is to be used, it is preferable to use the standard deviation of the IQ signal, and accordingly, a high reduction effect can be obtained with respect to a body motion having a large amplitude. In this case, the processing indicated by S3 in processing illustrated in FIG. 3 can be omitted.

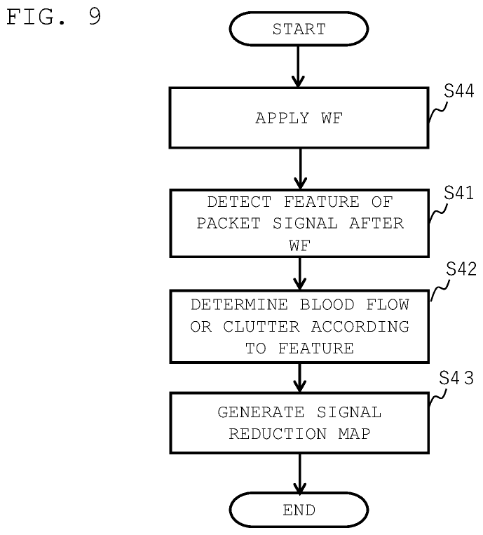

In the first embodiment, the clutter reduction processing executed by the clutter reducing unit 254 is executed before the processing executed by the filter unit 255 (S44 in FIG. 4). Alternatively, as illustrated in FIG. 9, the clutter reduction processing based on the features using the IQ signal and the velocity information may be executed after low-pass filtering executed by the filter unit 255.

What is claimed is:

1. An ultrasound imaging device comprising:
a transmission and reception unit configured to transmit and receive an ultrasonic wave; a clutter processing unit configured to remove a clutter signal from a reception signal of the ultrasonic wave received by the transmission and reception unit; and a color Doppler calculating unit configured to generate a color Doppler image using the reception signal from which the clutter signal is removed, wherein
the clutter processing unit includes a feature detecting unit configured to detect a feature of the reception signal, a determining unit configured to determine, based on the feature, whether the reception signal is a blood flow signal or a clutter signal, a reduction map generating unit configured to generate, based on a determination result, a reduction map for reducing the clutter signal, and a clutter reducing unit configured to apply the reduction map to the reception signal.

2. The ultrasound imaging device according to claim 1, wherein
the feature detecting unit detects, as the feature, a standard deviation of an IQ signal obtained by executing quadrature detection on the reception signal.

3. The ultrasound imaging device according to claim 1, wherein
the feature detecting unit sets, as the feature, a variance of a blood flow velocity calculated by the color Doppler calculating unit using an IQ signal before clutter removal processing.

4. The ultrasound imaging device according to claim 1, wherein
the feature detecting unit detects a plurality of features, and
the reduction map generating unit generates the reduction map by integrating determination results based on the plurality of features.

5. The ultrasound imaging device according to claim 4, wherein
the plurality of features include a standard deviation of an IQ signal obtained by executing quadrature detection on the reception signal and a variance of a blood flow velocity calculated using the reception signal before clutter removal processing.

6. The ultrasound imaging device according to claim 1, wherein
the determining unit generates a filter function using, as a cutoff value, a threshold value obtained by histogram analysis of the features, and determine the feature is a blood flow or a clutter.

7. The ultrasound imaging device according to claim 6, wherein
the determining unit generates, as the filter function, a plurality of filter functions having different characteristics.

8. The ultrasound imaging device according to claim 7, further comprising:
a display unit configured to allow a user to select a degree of clutter reduction processing that differs depending on a difference in the characteristics of the filter functions.

9. The ultrasound imaging device according to claim 1, wherein
the clutter processing unit further includes a filter unit that executes processing on the reception signal using a low-pass filter.

10. The ultrasound imaging device according to claim 9, wherein
the filter unit executes the processing using a low-pass filter on a reception signal in which clutter is reduced by the clutter reducing unit.

11. The ultrasound imaging device according to claim 9, wherein
the feature detecting unit detects the feature using the reception signal after the processing executed by the filter unit using the low-pass filter.

12. A method of generating a color Doppler image using a signal received by an ultrasonic wave transmission and reception unit of an ultrasound imaging device, the method comprising:
a clutter processing step of removing a clutter signal from a reception signal; and a color Doppler calculating step of generating a color Doppler image using the reception signal from which the clutter signal is removed, wherein
the clutter processing step includes a feature detecting step of detecting a feature of the reception signal, a determination step of determining, based on the feature, whether the reception signal is a blood flow signal or a clutter signal, a reduction map generating step of generating, based on a determination result, a reduction map for reducing the clutter signal, and a clutter reducing step of applying the reduction map to the reception signal.

13. The method of generating a color Doppler image according to claim 12, wherein
the feature includes a standard deviation of an IQ signal obtained by executing quadrature detection on the reception signal.

14. The method of generating a color Doppler image according to claim 12, further comprising:
a step of applying a low-pass filter to the reception signal after the reception signal is processed in the clutter reducing step.

15. The method of generating a color Doppler image according to claim 12, further comprising:
a step of applying a low-pass filter to the reception signal, wherein
the low-pass filter is applied to the reception signal before the feature detecting step.
